(19) United States
(12) Patent Application Publication
(10) Pub. No.: US 2006/0060324 A1
(43) Pub. Date: Mar. 23, 2006

(54) Strip casting system

(76) Inventor: Allan Stikeleather, South Easton, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/206,648

(22) Filed: Aug. 18, 2005

Related U.S. Application Data
(60) Provisional application No. 60/602,424, filed on Aug. 18, 2004.

(30) Foreign Application Priority Data
Aug. 18, 2005 (WO) PCT/US05/29937

(51) Int. Cl. A01H 5/02

(52) U.S. Cl. 164/47

(57) ABSTRACT

A method and apparatus for casting a metal strip by freezing a molten metal on an auger. The auger is arranged extending partially into the molten metal and the molten metal is allowed to at partially solidify on the auger. The auger is rotatable to convey the at least partially solidified molten metal axially along the auger and the strip of at least partially solidified molten metal is collected from the auger.

### CROSS-REFERENCE TO RELATED APPLICATIONS

This application claims the benefit of U.S. provisional patent application Ser. No. 60/602,424, filed Aug. 18, 2004, the entire disclosure of which is incorporated herein by reference.

### FIELD

The present invention relates to forming metal strips or sheets, and more particularly to casting metal strips from molten metal.

### BACKGROUND

Thin metallic strip stock material has been produced using a variety of processes. Often strip stock is produced by further processing an existing metallic product. According to one process, metallic wire or rod stock is pressed into a flat strip. Pressing wire or rod stock into a flat strip may conveniently be accomplished by passing the wire or rod stock through a series of rolls with successively decreasing spacing between the rolls to achieve a strip having a desired thickness. While rolling wire or rod stock is a convenient process, the final strip produced by such a process may exhibit work hardening, which may result in various undesirable mechanical characteristics.

According to another process, a metallic strip may be produced from a sheet metal stock. Strips may be formed from the sheet stock by cutting, slicing, or otherwise dividing the sheet. The sheet metal may be provided in a roll, allowing relatively long strips to be formed. Additionally, the sheet may be divided into several strips using simultaneous or sequential cutting operations.

Metallic strip material may also be produced directly. According to such an approach, the manufacture of the metallic strip does not rely on re-processing a stock material. One such directly forming process includes a twin roll casting process. In such a process a molten metallic material is supplied to rotating rolls. A strip of metal may be solidified at the rolls and passed between the rotating rolls. The cast strip may subsequently be processed by rolling, such as described above, to achieve a desired strip thickness.

### BRIEF DESCRIPTION OF THE DRAWINGS

Features and advantages of the present invention will be apparent from the following description of embodiments consistent therewith, which description should be considered in conjunction with the accompanying drawings, wherein:

FIG. 1 is a schematic illustration of an embodiment of a strip casting system consistent with the present disclosure;

FIG. 2 schematically depicts another embodiment of a strip casting system consistent with the present disclosure;

FIG. 3 is an end view of an auger disposed in a support tube consistent with the embodiment shown in FIG. 2; and

FIG. 4 schematically illustrates yet another embodiment of a strip casting system consistent with the present disclosure.

### DESCRIPTION

Generally the present disclosure relates to forming metal strips or sheets, and more particularly to casting metal strips from an at least partially molten metal. Consistent with the present disclosure, relatively thin metal strips may be cast in a generally continuous manner, enabling strips of varying lengths to be easily formed.

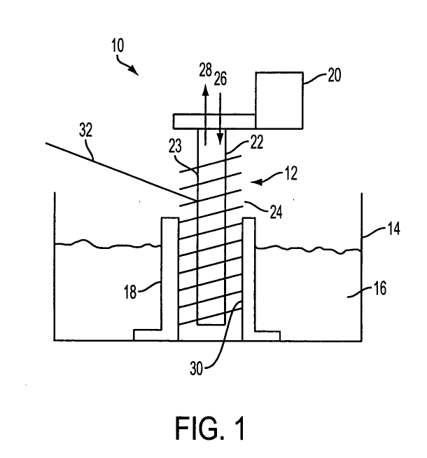

Referring to FIG. 1, an embodiment of a strip casting system 10 is schematically illustrated. As shown, the system may utilize an auger 12 extending into a tank 14 containing metal melt 16. The auger 12 may be disposed in a support tube 18. The support tube 18 may, in turn, be disposed in the tank 14 and may be in communication with the melt 16 such that a level of melt 16 may be maintained in the support tube 18 that may be generally the same as the level of the melt 16 in the tank 14. The auger 12 may be rotatably and translatably supported extending into the melt 16. The auger 12 may be coupled to a drive system 20 capable of rotating the auger 12.

The auger 12 may be configured as a longitudinal member 22 including at least one helical flight 24. The flight 24 may extend from an exterior surface of said longitudinal member 22. Alternatively, the flight 24 may be defined by a helical groove or slot defined in the longitudinal member 22. The auger 12 may be supported for rotational movement by one or more bearings, bushings, or similar features (not shown). As shown, the auger 12 may be coupled to a drive system 20 and the auger 12 may be rotationally driven by the drive system 20. The auger 12 may be directly coupled to the drive system 20, for example an output of the drive system 20 may be engaged with an input of the auger 12. Alternatively, the drive system 20 may be indirectly coupled to the auger 12, for example by a transmission or gear train, etc. including one or more drive and/or driven elements disposed between an output of the drive system 20 and an input of the auger 12.

In addition to being rotatably moveable within the support tube 18, the auger 12 may also be axially translatable relative to the metal melt 16 and/or with respect to the support tube 18, itself. Axial translation of the auger 12 may be accomplished using any of a variety of linear actuators or other suitable mechanisms. For example, the auger 12 may be coupled to one or more hydraulic rams or pistons capable of moving the auger 12 forward and backwards generally along the axis of the auger 12. Similarly, a screw drive system including a lead screw and a cooperating nut may be used to translate the auger 12 along the axis thereof. Various other mechanisms may also, or alternatively, be used to translate the auger 12. According to one embodiment, the auger 12 and the drive system 20 may be translatable together along an axis parallel to the axis of the auger 12. In such a configuration, both the auger 12 and the drive system 20 may move as a single unit, thereby allowing continued rotational motion of the auger 12 during translational movement. Alternatively, the auger 12 may be slidably coupled to the drive system 20, e.g., via a spline or similar feature. According to such an embodiment, the auger 12 may be rotationally driven by the drive system 20 and may simultaneously be axially translated independently of the drive system 20. Various other configurations and embodiments may suitably be employed herein.

The drive system 20 may include any of a variety of mechanisms capable of producing a rotational motion. For example, the drive system 20 may include an electric or a hydraulic motor or may include a gasoline or diesel engine, etc. According to one embodiment, the drive system 20 may be capable of driving the auger 12 at various suitable rotational speeds. Variable speed drive of the auger 12 may be accomplished by operating the motor or engine of the drive system 20 at different speeds. Alternatively, or additionally, variable speed drive of the auger 12 may be accomplished by varying a drive ratio between the drive system 20 and the auger 12. For example, the drive system 20 may be coupled to the auger 12 through a transmission capable of providing different drive ratios, e.g. by providing different gear ratios or different pulley ratios, in the case of a belt or chain drive. Similarly, the drive system 20 may drive the auger 12 at variable speeds by removing and/or replacing elements of the drive system 20 or power transfer system coupling the drive system 20 to the auger 12. For example, in an embodiment in which the drive system 20 is coupled to the auger 12 by a belt drive, the speed at which the auger 12 is driven may be varied by replacing either, or both, of the drive and the driven pulleys of the belt drive system to achieve a different pulley ratio. Variable speed operation of the auger 12 may also be achieved using a combination of a variable speed drive system 20 and a variable ratio coupling between the drive system 20 and the auger 12.

The auger 12 may also include a cooling system including a coolant inlet 26 and a coolant outlet 28 that may provide circulation of coolant through the auger 12. Consistent with the present disclosure, the coolant may be any suitable fluid, i.e., liquid or gaseous, heat transfer medium. According to one embodiment, the coolant may be a water-based or an oil-based heat transfer medium. According to one embodiment, the auger 12 may include an internal opening extending generally along at least a portion of the length of the auger 12. For example, the auger 12 may include a bore extending through at least a portion of the length of the auger 12. Circulation of coolant may be achieved by introducing coolant from the coolant inlet 26 into a first region of the bore, e.g. a region near the bottom of the bore, and exhausting the coolant to the coolant outlet 28 from a second region of the bore, e.g. from a region near the top of the bore. Alternatively, circulation of the coolant may be achieved using defined cooling passages defined within the auger 12. For example, the auger may include a coolant jacket or helically extending coolant passages. According to another embodiment, the auger 12 may include coolant passages defined in the flight 24 of the auger 12.

Similarly, the support tube 18 may also include a cooling system (not shown). As with the auger cooling system, the support tube 18 may include passages for circulating a liquid or gaseous heat transfer medium through the support tube 18. According to specific embodiments, the heat transfer medium may be a water or an oil based heat transfer medium, although various other liquid and/or gaseous heat transfer mediums may also suitable be used. Additionally, the outside of the support tube 18 may be at least partially thermally insulated from the melt 16. For example, the support tube 18 may include an insulating layer (not shown) surrounding the outside of the support tube 18. The support tube insulating layer may be provided formed from a material such as a ceramic material. Various other insulating materials may also suitably be employed. Thermally insulating the outside of the support tube 18 from the melt 16 may decrease the rate at which the cooling system of the support tube 18 draws heat from the melt 16 in the tank 14, thereby providing a more energy efficient system. Insulation may also be provided on the bottom of the auger 12 to prevent the metal melt 16 from solidifying on the bottom of the auger and blocking passage of the melt between the auger and the inside wall of the support tube 18.

The melt tank 14 may be formed from a material capable of withstanding the temperature of the metal melt 16. According to one embodiment, the melt tank 14 may include a heating system for melting solid or semi-molten metal contained in the tank 14. Alternatively, molten metal may be pored or pumped into the tank 14. The tank 14 may include a heating system for slowing the cooling rate of metal melt 16 contained within the tank 14 in order to prolong the time that the melt 16 remains in a fluid condition. According to an alternative embodiment, the melt tank 14 may not include a heating means, but may rather depend on the thermal mass and thermal inertia of the melt 16 contained therein to maintain the melt 16 in a fluid condition for a desired time. Still further, the melt tank 14 may be disposed in a furnace, etc., and thereby receive heat from an external source for melting and/or slowing the cooling rate of melt 16 contained in the tank 14.

According to one embodiment, a metal strip 32 may be cast from the melt 16 contained by the tank 14 by at least partially solidifying metal melt 16 on the root 23 of the auger 12 in between the flights 24. As used herein, the root 23 of the auger 12 is the surface of the auger 12 in between adjacent flights 24. The root 23 may be parallel to the axis of the auger 12, or may be at an angle, thereby forming a taper. The root 23 may also include one or more regions that are tapered and one or more regions that are parallel to the axis of the auger 12. In an example of an auger 12 including a flight extending from a longitudinal member 22, the root 23 may present a helical portion of the longitudinal member exposed between adjacent flights 24. As illustrated, the auger 12 may be disposed in the support tube 18 such that at least a portion of the auger 12 is below the level of the melt 16. The support tube 18 may include one or more openings thereby allowing the metal melt 16 to flow into the support tube 18 and obtain a level that may be generally the same as the level of the metal melt 16 in the tank 14.

Coolant may be circulated through the auger 12 via the inlet 26 and outlet 28. The circulating coolant may maintain at least a portion of the root 23 of the auger 12 at a temperature that is below the freezing point of the metal melt 16 in the tank 14. Accordingly, when the metal melt 16 contacts the root 23 of the auger 12, the metal melt 16 may freeze, i.e., at least partially solidify, on the root 23 of the auger 12, and form a strip 32. The auger 12 may be rotated via the drive system 20 to convey the strip 32 up the auger 12. The strip 32 may be collected from the auger 12 at a point after which the melt 16 has at least partially solidified to form a strip 32, for example after the strip 32 emerges from the top of the support tube 18. The strip 32 may be conveyed up the auger 12 as a result of the interaction between the strip 32, the root 23 of the auger 12, the flight 24 of the auger 12, and the inner surface 30 of the support tube 18.

The thickness of the strip formed on the auger 12 may be related to a number of variables and characteristics. For example, the thickness of the strip 32 formed on the auger 12 may be a function of the cooling capacity of the auger cooling system and the magnitude of the temperature of the melt 16 above the freezing point of the melt 16, i.e., the amount of heat that must be removed to freeze a layer of melt 16 into a strip 32 on the auger 12, and the rotational speed of the auger 12. The slope of the flight 24, as well as the pitch, depth of the flight, and finish of the flight land and of the root 23 may also affect the thickness of the strip of frozen metal melt 16 deposited on the flight 24. For example, at a given rotational speed, a smaller pitch may increase the residence time of an individual segment of metal melt 16 frozen on the auger 12. The thickness of the strip may differ depending upon the cooling capacity of the cooling system, the rotational speed of the auger, and the distance between the inside of the support tube and the depth of the flights 24.

The uniformity of the strip 32 may be related to the tolerances of the various components of the system 10. For example, a narrow tolerance of the depth of the flights 24 may provide a uniform thickness of frozen melt carried by the auger 12. Similarly, a narrow clearance tolerance between the auger 12 and the inside wall 30 of the support tube may provide a uniform thickness of frozen melt extending beyond the diameter of the auger 12. In a related manner, the uniformity of the strip 32 may be related to the concentricity of the auger 12 and of the support tube 18, as well as the concentricity of alignment between the auger 12 and the support tube 18.

The characteristics of the auger 12 and the support tube 18 may be adjusted to achieve a desired frictional interaction between metal melt 16 freezing on the auger 12 and the support tube 18. According to one aspect, the inner surface 30 of the support tube 18 may be smooth to promote the ease of passage of metal melt 16 frozen on the auger 12, that is, to reduce drag between the metal strip 32 and the inner surface 30 of the support tube 18. Similarly, the flight angle, pitch, finish and land depth may be provided to achieve a desired level of friction between the strip of metal melt 16 frozen on the root 23 of the auger 12, the flight 24 of the auger 12, and the inner surface 30 of the support tube 18.

Consistent with one aspect, the auger 12 may be moveable in and out of the outer tube 18. Movement of the auger 12 in and out of the support tube 18 may be achieved either with the drive system 20, or independently of the drive system 20. Movement of the auger 20 in and out of the support tube 18 may be coordinated with the rotational speed of the auger 12 and the pitch of the flight 24 to accommodate the freezing rate of the metal melt 16 to form the strip 32 having a desired thickness.

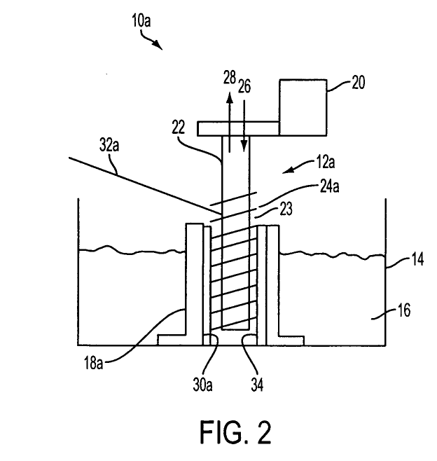

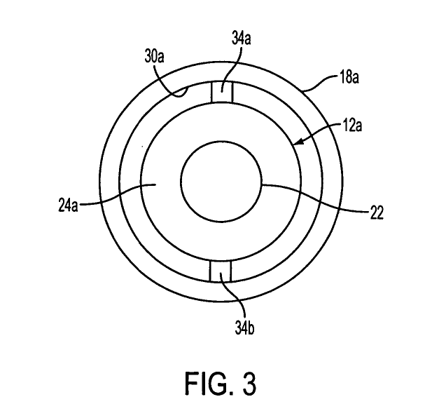

With reference to FIGS. 2 and 3, a strip casting system 10a may be provided having a similar configuration as the strip casting system 10 illustrated in FIG. 1. According to one aspect, the support tube 18a may advantageously include one or more metal stops 34a, 34b extending inwardly from the inner wall 30a. A greater or fewer number of metal stops may be used consistent with the disclosed system. The outside diameter of the helical flight 24a of the auger may be sized to be received between the metal stops 34a, 34b with a slight clearance to permit rotation of the auger in between the stops 34a, 34b. Accordingly, the auger may rotate in between the metal stops 34a, 34b without scraping against the metal stops 34a, 34b. While the edges of the stops 34a, 34b adjacent the auger 12a are shown in FIG. 3 having a circular shape conforming to the profile of the auger 12a, the edges of the stops 34a, 34b may be provided having other shapes, including a squared-off shape.

In operation, the auger 12a may be moved into the metal melt 16 inside of the support tube 18a and positioned between the metal stops 34a, 34b. With the auger 12 extending into the melt 16, the auger 12a may be cooled, thereby causing metal melt 16 to freeze on the root 23 of the auger 12a. As the melt 16 is freezing on the auger 12a, the auger 12a may also be rotated by the drive system 20. The stops 34a, 34b may prevent the freezing melt from rotating with the auger 12a. Instead, the interaction of the melt 16 freezing on the auger 12a and the stops 34a, 34b may drive the metal up the auger 12a. As the frozen melt 16 on the auger 12a is driven above the surface of the melt 16, and or the top of the support tube 18a, the frozen melt 16 may be collected as a strip 32a.

According to another embodiment, an auger including a helical flight, similar to the auger described previously, may be oriented parallel to the surface of the metal melt. The auger may be positioned so that at least a portion of the flight extends into the liquid metal melt. According to one embodiment, the flight of the auger may extend into the metal melt at least a portion of the depth of the flight. Coolant circulating through the auger may reduce the temperature of the auger to a temperature below the freezing point of the metal melt. Accordingly, liquid melt may freeze, or solidify, on a portion of the root of the auger extending into the liquid melt. The auger may be rotated by a drive system, whereby a region of the auger root may extend in to the metal melt for an angular portion of the rotation of the auger and may be disposed above the metal melt for an angular portion of the rotation of the auger. A strip of frozen melt may, accordingly, be collected from a region of the auger root disposed above the metal melt as the auger is rotated.

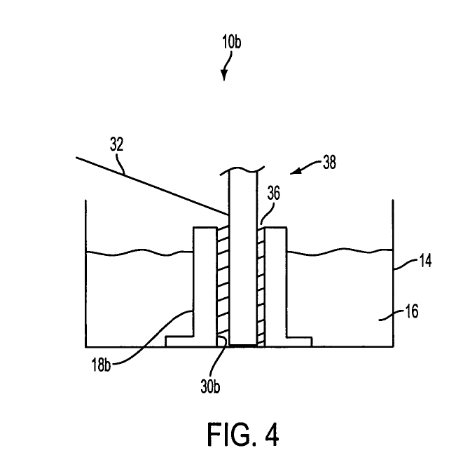

Turning to FIG. 4, yet another embodiment of a strip casting system 10b is illustrated. The strip casting system 10b may include a support tube 18b having a threaded inner wall 30b including at least one inwardly extending helical flight 36. A longitudinal member 38 having a circular cross-section may be received at least partially within the inside diameter of the helical flight 36. The longitudinal member 38 and the threaded inner wall 30b may be rotatably driven relative to one-another. For example, the longitudinal member 38 may be rotationally fixed, and the support tube 18b may be rotationally driven around the longitudinal member 38. Alternatively, the support tube 18b may be rotationally fixed and the longitudinal member 38 may be rotationally driven. According to another variation, both the support tube 18b and the longitudinal member 38 may be rotationally driven. For example, the support tube 18b and longitudinal member 38 may be counter rotated. Alternatively, the support tube 18b and the longitudinal member 38 may be driven at different rotational speeds and in the same direction. The different rotational speeds of the longitudinal member 38 and the support tube 18b may provide a net, or effective, rotation of the longitudinal member 38 relative to the support tube 18b.

Similar to previously described embodiments, the longitudinal member 38 may be positioned within the support tube 18b and extending into the metal melt 16. One or both of the longitudinal member 38 and the support tube 18b may cooled, thereby causing metal melt to freeze, or at least partially solidify, on the longitudinal member 38 and/or the inside surface 30b of the support tube 18b. As the melt 16 freezes, the longitudinal member 38 and the support tube 18b may be rotated relative to one another. The interaction of the frozen melt 16 with the surface of the longitudinal member and the inside surface 30b, and/or flight 36, of the support tube 18b may drive the frozen melt 16 up the support tube 18b or longitudinal member 38. The frozen melt 16 may then be collected from the strip casting system 10b as it emerges as a strip 32.

Still further embodiments of strip casting systems consistent with the present disclosure may be provided in which various features of the preceding embodiments may advantageously combined. For example, an embodiment may be provided including an auger with one or more flights that is received in a support tube having a threaded inner surface. At least a portion of the support tube and the auger may be disposed in a metal melt. One or both of the support tube and the auger may be cooled causing at least a portion of the metal melt disposed between the auger and the support tube to at least partially solidify. One, or both, of the auger and the support tube may be rotationally driven. According to one specific embodiment, the auger and the support tube may be driven to counter rotate relative to one another. According to still another embodiment, the auger and the support tube may be co-rotate, but at different rotational speeds, thereby producing a net, or effective, rotation of the auger relative to the support tube. The interaction of the at least partially solidified melt with the support tube and the auger may cause the at least partially solidified melt to be driven up the auger and/or the support tube. As with previous embodiments, the at least partially solidified melt may be collected as a strip as it emerges from the support tube.

Herein above, the strip casting system of the present disclosure has been discussed in terms of casting a metal strip from a liquid metal melt. The system herein, however, may suitably be used for casting a strip from materials other than metal. The system herein may be used to cast strips of any material that may be at least partially solidified from a liquid between the support tube and auger or longitudinal member.

The described embodiments herein are susceptible to numerous variations and modifications, including the combination of various aspects and features disclosed herein, without departing from the primary principles thereof. Accordingly, the described embodiments should not be considered to be limiting on the scope of the invention herein.

What is claimed is:

1. A strip casting apparatus comprising:
a tank configured to contain metal melt;
a rotatable auger extending at least partially into said metal melt; and
a support tube in communication with said metal melt, said auger at least partially disposed in said support tube.

2. The apparatus according to claim 1, further comprising a drive system coupled to said auger for rotatably driving said auger.

3. The apparatus according to claim 1, wherein said auger comprise a longitudinal member including at least one helical flight.

4. The apparatus according to claim 1, wherein said auger is axially translatable.

5. The apparatus according to claim 2, wherein said drive system is indirectly coupled to said auger.

6. The apparatus according to claim 5, wherein said drive system is coupled to said auger via a transmission.

7. The apparatus according to claim 1, wherein said auger comprises a cooling system.

8. The apparatus according to claim 1, wherein said support tube comprises a cooling system.

9. The apparatus according to claim 1, wherein said support tube comprises at least one inwardly extending stop.

10. A method of casting a metal strip comprising:
disposing at least a portion of an auger in an at least partially molten metal;
at least partially solidifying said molten metal on said auger; and
collecting said at least partially solidified metal from said auger.

11. The method according to claim 10, wherein disposing at least a portion of said auger in said at least partially molten metal comprises disposing at least a portion of said auger in a support tube, said support tube being in communication with said molten metal.

12. The method according to claim 10, further comprising rotating said auger.

13. The method according to claim 10, further comprising conveying said strip axially along said auger.

14. The method according to claim 10, further comprising cooling said auger.

15. A strip casting apparatus comprising:
a support tube in communication with an at least partially molten metal, said support tube comprising a threaded inner wall;
a longitudinal member received at least partially within said support tube;
said support tube and said longitudinal member being rotatable relative to one another.

16. The apparatus according to claim 15, wherein said longitudinal member is rotationally fixed, and said support tube is rotatable around said longitudinal member.

17. The apparatus according to claim 15, wherein said support tube is rotationally fixed and said longitudinal member is rotatable.

18. The apparatus according to claim 15, wherein said longitudinal member and said support tube are rotatable.

19. The apparatus according to claim 18, wherein said longitudinal member is rotatable at a first speed and said support tube is rotatable at a second speed.

20. The apparatus according to claim 15, wherein at least one of said longitudinal member and said support tube is cooled.
